(19) United States
(12) Patent Application Publication
(10) Pub. No.: US 2024/0403342 A1
(43) Pub. Date: Dec. 5, 2024

(54) INFORMATION PROCESSING APPARATUS, INFORMATION PROCESSING METHOD, AND INFORMATION PROCESSING PROGRAM

(71) Applicant: FUJIFILM CORPORATION, Tokyo (JP)

(72) Inventor: Yu HASEGAWA, Tokyo (JP)

(21) Appl. No.: 18/805,545

(22) Filed: Aug. 15, 2024

Related U.S. Application Data
(63) Continuation of application No. PCT/JP2023/005843, filed on Feb. 17, 2023.

(30) Foreign Application Priority Data
Feb. 18, 2022 (JP) 2022-024250

(51) Int. Cl. G06F 16/33; G06F 16/35

(52) U.S. Cl. CPC G06F 16/3344; G06F 16/3334; G06F 16/353

(57) ABSTRACT

An information processing apparatus including at least one processor, wherein the processor is configured to: extract at least one word included in a comment on findings; classify the word into a predetermined item; display the word for each item on a display; and search for medical information similar to content of the comment on findings from among plural pieces of recorded medical information based on the word.

### CROSS REFERENCE TO RELATED APPLICATIONS

This application is a continuation of International Application No. PCT/JP2023/005843, filed on Feb. 17, 2023, which claims priority from Japanese Patent Application No. 2022-024250, filed on Feb. 18, 2022. The entire disclosure of each of the above applications is incorporated herein by reference.

### BACKGROUND

### Technical Field

The present disclosure relates to an information processing apparatus, an information processing method, and an information processing program.

### Related Art

In the related art, image diagnosis is performed using medical images obtained by imaging apparatuses such as computed tomography (CT) apparatuses and magnetic resonance imaging (MRI) apparatuses. Further, medical images are analyzed via computer aided detection/diagnosis (CAD) using a discriminator in which learning is performed by deep learning or the like, and regions of interest including structures, lesions, and the like included in the medical images are detected and/or diagnosed. The medical images and analysis results via CAD are transmitted to a terminal of a healthcare professional such as a radiologist who interprets the medical images. The healthcare professional such as a radiologist interprets the medical image by referring to the medical image and analysis result using his or her own terminal and creates an interpretation report.

In addition, various methods have been proposed to support the creation of interpretation reports in order to reduce the burden of the interpretation work of a radiologist. For example, JP2019-153250A discloses a technology for creating an interpretation report based on a keyword input by a radiologist and an analysis result of a medical image. In the technology disclosed in JP2019-153250A, a sentence to be included in the interpretation report is created by using a recurrent neural network trained to generate a sentence from input characters.

Further, since the creation work of the interpretation report may be performed while referring to past similar cases, various methods related to searching for similar cases have been proposed. For example, WO2012/104949A discloses a technology for extracting keywords from image findings included in interpretation information (an interpretation report) and calculating a text similarity degree with an interpretation report registered in a case database. In addition, for example, JP2017-021648A discloses a technology for searching for a plurality of reports including input characters from a report database and extracting and displaying a sentence including the characters from each report.

In recent years, in searching for similar cases, there has been a desire for a technology that can more easily search for cases.

### SUMMARY

The present disclosure provides an information processing apparatus, an information processing method, and an information processing program capable of easily searching for cases.

According to a first aspect of the present disclosure, there is provided an information processing apparatus comprising at least one processor, in which the processor is configured to: extract at least one word included in a comment on findings; classify the word into a predetermined item; display the word for each item on a display; and search for medical information similar to content of the comment on findings from among a plurality of pieces of recorded medical information based on the word.

According to a second aspect of the present disclosure, in the first aspect, the processor may be configured to, in a case in which a plurality of the words are extracted from the comment on findings, receive selection of at least one of the words used for searching for the medical information.

According to a third aspect of the present disclosure, in the second aspect, the processor may be configured to display an operation unit that receives the selection of at least one of the words used for searching for the medical information on the display in association with the word.

According to a fourth aspect of the present disclosure, in any one of the first to third aspects, the processor may be configured to search for the medical information based on at least one of a synonym or a related word predetermined for each word.

According to a fifth aspect of the present disclosure, in the fourth aspect, the processor may be configured to, in a case in which the number of pieces of the medical information found based on the word does not satisfy a predetermined threshold value, search for the medical information based on the synonym.

According to a sixth aspect of the present disclosure, in the fifth aspect, the processor may be configured to, in a case in which the number of pieces of the medical information found based on the synonym does not satisfy a predetermined threshold value, search for the medical information based on the related word.

According to a seventh aspect of the present disclosure, in any one of the fourth to sixth aspects, the processor may be configured to specify a plurality of words whose degree of co-occurrence is equal to or higher than a predetermined threshold value, based on the plurality of pieces of medical information, as related words.

According to an eighth aspect of the present disclosure, in any one of the first to seventh aspects, the processor may be configured to: acquire a plurality of the comments on findings; and receive selection of at least one of the plurality of comments on findings used for searching for the medical information.

According to a ninth aspect of the present disclosure, in the eighth aspect, the processor may be configured to: in a case in which the number of pieces of the medical information found based on the word does not satisfy a predetermined threshold value, extract at least one word included in another comment on findings related to the comment on findings selected from among the plurality of comments on findings; and search for the medical information based on the word extracted from the other comment on findings.

According to a tenth aspect of the present disclosure, in any one of the first to ninth aspects, the processor may be configured to search for the medical information based on a weight predetermined for each item.

According to an eleventh aspect of the present disclosure, in the tenth aspect, the processor may be configured to receive a setting of the weight for each item.

According to a twelfth aspect of the present disclosure, in any one of the first to eleventh aspects, the processor may be configured to: specify factuality of the extracted word; and search for the medical information based on the factuality.

According to a thirteenth aspect of the present disclosure, in any one of the first to twelfth aspects, the processor may be configured to, in a case in which the word indicates a numerical value, receive designation of a numerical value range that is considered to be similar to the comment on findings.

According to a fourteenth aspect of the present disclosure, in any one of the first to thirteenth aspects, the word may indicate information regarding an abnormal shadow in a medical image, and the item may indicate at least one of a name, a property, a disease name, a position, or a measured value of the abnormal shadow.

According to a fifteenth aspect of the present disclosure, in any one of the first to fourteenth aspects, the processor may be configured to display the found medical information on the display.

According to a sixteenth aspect of the present disclosure, in any one of the first to fifteenth aspects, the processor may be configured to: derive a degree of similarity between the content of the comment on findings and the medical information; and display the derived degree of similarity on the display.

According to a seventeenth aspect of the present disclosure, in any one of the first to sixteenth aspects, the medical information may indicate at least one of a medical image, a comment on findings regarding the medical image, subject information regarding a subject of the medical image, or biological information acquired from the subject.

According to an eighteenth aspect of the present disclosure, in the seventeenth aspect, the medical information may indicate the comment on findings.

According to a nineteenth aspect of the present disclosure, there is provided an information processing method comprising: extracting at least one word included in a comment on findings; classifying the word into a predetermined item; displaying the word for each item on a display; and searching for medical information similar to content of the comment on findings from among a plurality of pieces of recorded medical information based on the word.

According to a twentieth aspect of the present disclosure, there is provided an information processing program for causing a computer to execute a process comprising: extracting at least one word included in a comment on findings; classifying the word into a predetermined item; displaying the word for each item on a display; and searching for medical information similar to content of the comment on findings from among a plurality of pieces of recorded medical information based on the word.

The information processing apparatus, the information processing method, and the information processing program according to the aspects of the present disclosure can easily search for cases.

### BRIEF DESCRIPTION OF THE DRAWINGS

FIG. 1 is a diagram showing an example of a schematic configuration of an information processing system.

FIG. 2 is a diagram showing an example of structured data.

FIG. 3 is a block diagram showing an example of a hardware configuration of an information processing apparatus.

FIG. 4 is a block diagram showing an example of a functional configuration of the information processing apparatus.

FIG. 5 is a diagram showing an example of a screen displayed on a display.

FIG. 6 is a diagram showing an example of a screen displayed on a display.

FIG. 7 is a diagram showing an example of words.

FIG. 8 is a diagram showing an example of structured data.

FIG. 9 is a diagram showing an example of a screen displayed on a display.

FIG. 10 is a diagram showing an example of a screen displayed on a display.

FIG. 11 is a diagram showing an example of a screen displayed on a display.

FIG. 12 is a flowchart showing an example of information processing.

FIG. 13 is a diagram showing an example of a screen displayed on a display.

### DETAILED DESCRIPTION

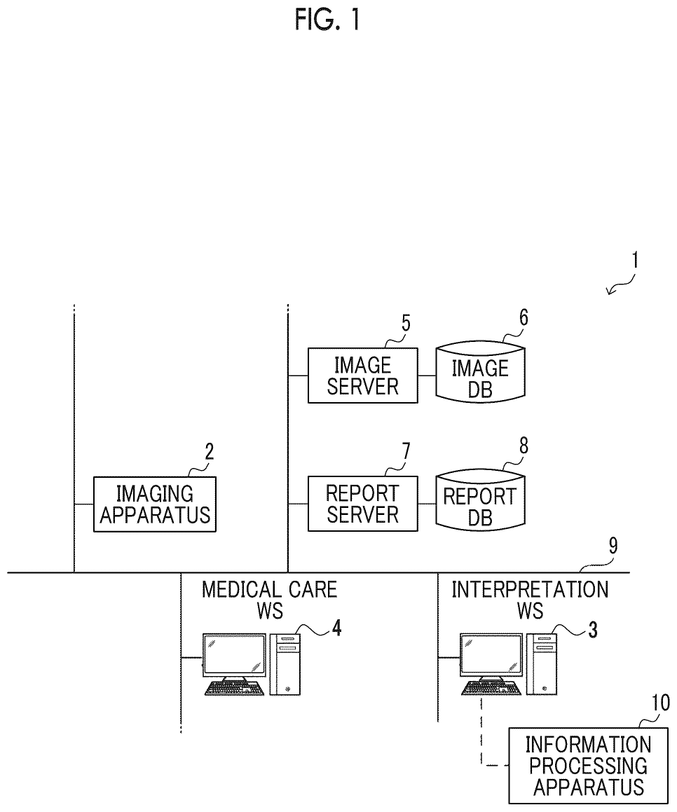

Hereinafter, an exemplary embodiment of the present disclosure will be described with reference to the drawings. First, a configuration of an information processing system 1 to which an information processing apparatus of the present disclosure is applied will be described. FIG. 1 is a diagram showing a schematic configuration of the information processing system 1. The information processing system 1 shown in FIG. 1 performs imaging of an examination target part of a subject and storing of a medical image acquired by the imaging based on an examination order from a doctor in a medical department using a known ordering system. In addition, the information processing system 1 performs an interpretation work of a medical image and creation of an interpretation report by a radiologist and viewing of the interpretation report by a doctor of a medical department that is a request source.

As shown in FIG. 1, the information processing system 1 includes an imaging apparatus 2, an interpretation work station (WS) 3 that is an interpretation terminal, a medical care WS 4, an image server 5, an image database (DB) 6, a report server 7, and a report DB 8. The imaging apparatus 2, the interpretation WS 3, the medical care WS 4, the image server 5, the image DB 6, the report server 7, and the report DB 8 are connected to each other via a wired or wireless network 9 in a communicable state.

Each apparatus is a computer on which an application program for causing each apparatus to function as a component of the information processing system 1 is installed. The application program may be recorded on, for example, a recording medium, such as a digital versatile disc (DVD) or a compact disc read-only memory (CD-ROM), and distributed, and be installed on the computer from the recording medium. In addition, the application program may be stored in, for example, a storage device of a server computer connected to the network 9 or in a network storage in a state in which it can be accessed from the outside, and be downloaded and installed on the computer in response to a request.

The imaging apparatus 2 is an apparatus (modality) that generates a medical image T showing a diagnosis target part of the subject by imaging the diagnosis target part. Specifically, examples of the imaging apparatus 2 include a simple X-ray imaging apparatus, a CT apparatus, an MRI apparatus, a positron emission tomography (PET) apparatus, and the like. The medical image generated by the imaging apparatus 2 is transmitted to the image server 5 and is stored in the image DB 6.

The interpretation WS 3 is a computer used by, for example, a user such as a radiologist of a radiology department to interpret a medical image and to create an interpretation report, and encompasses an information processing apparatus 10 according to the present exemplary embodiment. In the interpretation WS 3, a viewing request for a medical image to the image server 5, various types of image processing for the medical image received from the image server 5, display of the medical image, and input reception of a sentence regarding the medical image are performed. In the interpretation WS 3, analysis processing for medical images, support for creating an interpretation report based on the analysis result, a registration request and a viewing request for the interpretation report to the report server 7, and display of the interpretation report received from the report server 7 are performed. The above processes are performed by the interpretation WS 3 executing software programs for respective processes.

The medical care WS 4 is a computer used by, for example, a user such as a doctor in a medical department to observe a medical image in detail, view an interpretation report, create an electronic medical record, and the like, and is configured to include a processing device, a display device such as a display, and an input device such as a keyboard and a mouse. In the medical care WS 4, a viewing request for the medical image to the image server 5, display of the medical image received from the image server 5, a viewing request for the interpretation report to the report server 7, and display of the interpretation report received from the report server 7 are performed. The above processes are performed by the medical care WS 4 executing software programs for respective processes.

The image server 5 is a general-purpose computer on which a software program that provides a function of a database management system (DBMS) is installed. The image server 5 is connected to the image DB 6. The connection form between the image server 5 and the image DB 6 is not particularly limited, and may be a form connected by a data bus, or a form connected to each other via a network such as a network attached storage (NAS) and a storage area network (SAN).

The image DB 6 is realized by, for example, a storage medium such as a hard disk drive (HDD), a solid-state drive (SSD), and a flash memory. In the image DB 6, the medical image acquired by the imaging apparatus 2 and accessory information attached to the medical image are registered in association with each other.

The accessory information may include, for example, identification information such as an image identification (ID) for identifying a medical image, a tomographic ID assigned to each tomographic image included in the medical image, a subject ID for identifying a subject, and an examination ID for identifying an examination. In addition, the accessory information may include, for example, imaging information related to imaging such as an imaging method, an imaging condition, and an imaging date and time related to imaging of a medical image. The “imaging method” and “imaging condition” are, for example, a type of the imaging apparatus 2, an imaging part, an imaging protocol, an imaging sequence, an imaging method, the presence or absence of use of a contrast medium, a slice thickness in tomographic imaging, and the like. In addition, the accessory information may include subject information related to the subject such as the name, date of birth, age, and gender of the subject.

In a case in which the image server 5 receives a request to register a medical image from the imaging apparatus 2, the image server 5 prepares the medical image in a format for a database and registers the medical image in the image DB 6. In addition, in a case in which the viewing request from the interpretation WS 3 and the medical care WS 4 is received, the image server 5 searches for a medical image registered in the image DB 6 and transmits the found medical image to the interpretation WS 3 and to the medical care WS 4 that are viewing request sources.

The report server 7 is a general-purpose computer on which a software program that provides a function of a database management system is installed. The report server 7 is connected to the report DB 8. The connection form between the report server 7 and the report DB 8 is not particularly limited, and may be a form connected by a data bus or a form connected via a network such as a NAS and a SAN.

The report DB 8 is realized by, for example, a storage medium such as an HDD, an SSD, and a flash memory. In the report DB 8, an interpretation report created in the interpretation WS 3 is registered.

Further, in a case in which the report server 7 receives a request to register the interpretation report from the interpretation WS 3, the report server 7 prepares the interpretation report in a format for a database and registers the interpretation report in the report DB 8. Further, in a case in which the report server 7 receives the viewing request for the interpretation report from the interpretation WS 3 and the medical care WS 4, the report server 7 searches for the interpretation report registered in the report DB 8, and transmits the found interpretation report to the interpretation WS 3 and to the medical care WS 4 that are viewing request sources.

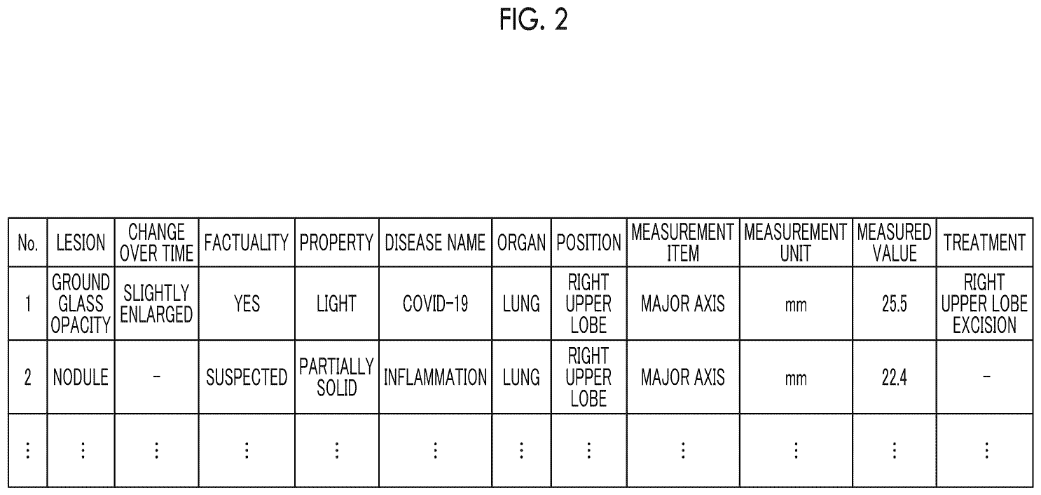

In addition, the report server 7 may extract words from comments on findings included in the interpretation report registered in the report DB 8, classify the extracted words into predetermined items (so-called “structuring”), and store structured data (details will be described later) in the report DB 8. FIG. 2 shows an example of structured data.

The network 9 is, for example, a network such as a local area network (LAN) and a wide area network (WAN). The imaging apparatus 2, the interpretation WS 3, the medical care WS 4, the image server 5, the image DB 6, the report server 7, and the report DB 8 included in the information processing system 1 may be disposed in the same medical institution, or may be disposed in different medical institutions or the like. Further, the number of each apparatus of the imaging apparatus 2, the interpretation WS 3, the medical care WS 4, the image server 5, the image DB 6, the report server 7, and the report DB 8 is not limited to the number shown in FIG. 1, and each apparatus may be composed of a plurality of apparatuses having the same functions.

Next, the information processing apparatus 10 according to the present exemplary embodiment will be described. The information processing apparatus 10 has a function of searching, based on a certain comment on findings, for a past case similar to the content of comment on findings. The case is, for example, an interpretation report, a comment on findings, and structured data (see FIG. 2) recorded in the report DB 8. A case is an example of medical information according to the embodiment of the present disclosure. As described above, the information processing apparatus 10 is encompassed in the interpretation WS 3.

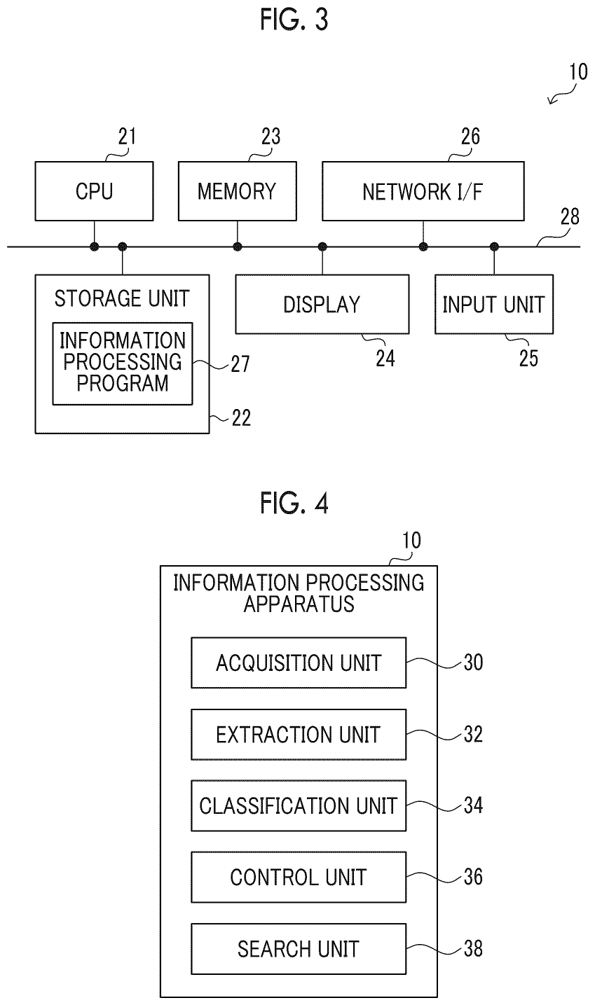

First, with reference to FIG. 3, an example of a hardware configuration of the information processing apparatus 10 according to the present exemplary embodiment will be described. As shown in FIG. 3, the information processing apparatus 10 includes a central processing unit (CPU) 21, a non-volatile storage unit 22, and a memory 23 as a temporary storage area. Further, the information processing apparatus 10 includes a display 24 such as a liquid crystal display, an input unit 25 such as a keyboard and a mouse, and a network interface (I/F) 26. The network I/F 26 is connected to the network 9 and performs wired or wireless communication. The CPU 21, the storage unit 22, the memory 23, the display 24, the input unit 25, and the network I/F 26 are connected to each other via a bus 28 such as a system bus and a control bus so that various types of information can be exchanged.

The storage unit 22 is realized by, for example, a storage medium such as an HDD, an SSD, and a flash memory. An information processing program 27 in the information processing apparatus 10 is stored in the storage unit 22. The CPU 21 reads out the information processing program 27 from the storage unit 22, loads the read-out program into the memory 23, and executes the loaded information processing program 27. The CPU 21 is an example of a processor of the present disclosure. As the information processing apparatus 10, for example, a personal computer, a server computer, a smartphone, a tablet terminal, a wearable terminal, or the like can be applied as appropriate.

Next, with reference to FIG. 4, an example of a functional configuration of the information processing apparatus 10 according to the present exemplary embodiment will be described. As shown in FIG. 4, the information processing apparatus 10 includes an acquisition unit 30, an extraction unit 32, a classification unit 34, a control unit 36, and a search unit 38. In a case in which the CPU 21 executes the information processing program 27, the CPU 21 functions as the acquisition unit 30, the extraction unit 32, the classification unit 34, the control unit 36, and the search unit 38.

The acquisition unit 30 acquires, from the image server 5, at least one medical image for which an interpretation report is to be created. Note that the acquisition unit 30 may acquire, for example, a plurality of medical images related to the same subject, such as a CT image consisting of a plurality of tomographic images and a plurality of medical images (for example, a combination of a simple CT image, a contrast CT image, and an MRI image) having different types of imaging apparatuses 2, imaging conditions, and imaging methods. In addition, the acquisition unit 30 may acquire accessory information attached to the medical image.

Further, the acquisition unit 30 may acquire biological information acquired from the subject of the medical image to be the target of creating the interpretation report. The biological information may be, for example, information indicating at least one of a body temperature, a heart rate, an electrocardiogram, an electromyogram, a blood pressure, an arterial blood oxygen saturation (SpO2), a blood glucose level, a lipid level, or the like. Further, for example, the biological information may be information indicating at least one result of various tests such as a hematological test, a biochemical test, a pathological test, an immunological test, a genetic test, a bacterial test, a urine test, and an infectious disease test. The biological information may be stored in advance in the storage unit 22, for example, or may be appropriately acquired from the image server 5 (image DB 6), the report server 7 (report DB 8), and other external devices (not shown).

The hematological test is a test for obtaining, for example, a leukocyte count, an erythrocyte count, and a hemoglobin concentration as a test result. The biochemical test is a test for obtaining various indicators related to, for example, enzymes, proteins, glucose, lipids, and electrolytes as a test result. The pathological test is a test for obtaining, for example, the presence or absence, the type, and the like of a lesion specified by observing cells, living body tissues, and the like collected from a subject are obtained as a test result. The immunological test is a test for obtaining, for example, a detection result of a substance peculiar to a tumor marker, a hormone, an allergy, or the like as a test result.

The genetic test is a test for obtaining, for example, genetic information related to a constitution, a disease, or the like as a test result by analyzing deoxyribonucleic acid (DNA). The bacterial test is a test for obtaining, for example, a type and an amount of bacteria present in a body, on a surface of the body surface, or the like as a test result. The urine test is a test for obtaining, for example, glucose in urine, protein in urine, and occult blood in urine as a test result. The infectious disease test is a test for obtaining, for example, presence or absence of infection caused by various infectious diseases such as influenza infection and novel coronavirus infection as a test result.

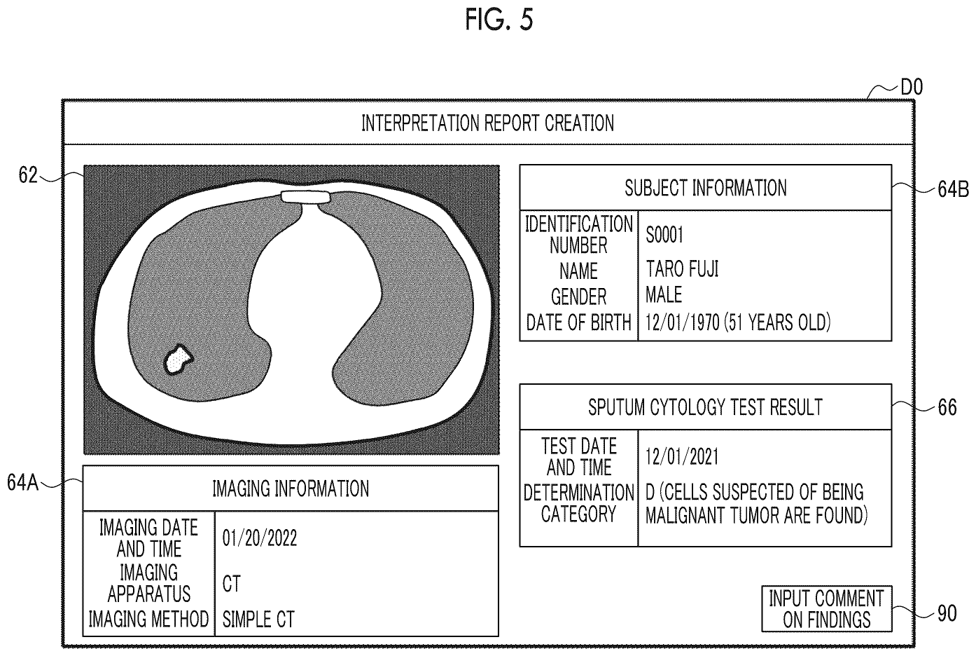

The control unit 36 performs control to display the medical image, accessory information, and biological information acquired by the acquisition unit 30 on the display 24. FIG. 5 shows an example of a screen DO displayed on the display 24 by the control unit 36. The screen DO includes a medical image 62 acquired by the acquisition unit 30, imaging information 64A and subject information 64B as an example of the accessory information, and a test result 66 of sputum cytology as an example of the biological information.

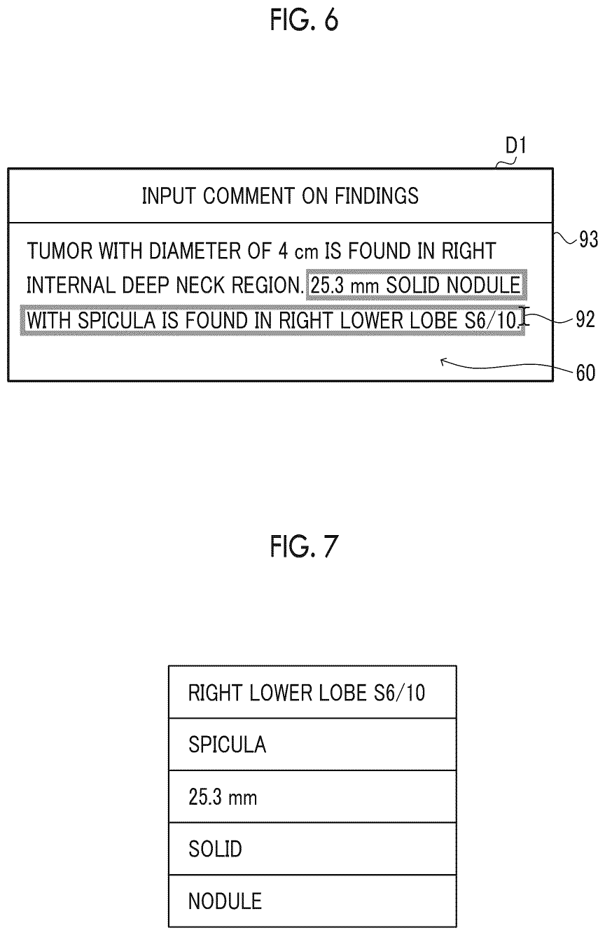

Further, the screen DO includes a comment-on-findings input button 90. In a case in which the comment-on-findings input button 90 is selected by the operation of the input unit 25 by the user, the control unit 36 performs control to display a screen D1 shown in FIG. 6 on the display 24. The screen D1 includes a text box 93 for receiving an input of a comment on findings 60. The control unit 36 receives an input of at least one comment on findings 60 by the user through the text box 93.

Note that, as shown in FIG. 6, a plurality of comments on findings may be input to the text box 93. In a case in which a plurality of comments on findings are acquired in this way, the control unit 36 may receive selection of at least one comment on findings used for searching for a case from among the plurality of comments on findings. For example, the control unit 36 may receive selection of some of the plurality of comments on findings input to the text box 93 by an operation of a pointer 92 by the user via the input unit 25. In FIG. 6, the selected comment on findings is shaded.

The acquisition unit 30 acquires at least one comment on findings received on the screen D1. In the example in FIG. 6, the acquisition unit 30 acquires the selected comment on findings, “A 25.3 mm solid nodule with a spicula is found in the right lower lobe S6/10”, from among the plurality of comments on findings input to the text box 93.

The extraction unit 32 extracts at least one word included in the comment on findings acquired by the acquisition unit 30. FIG. 7 shows a list of words extracted from the comment on findings selected in FIG. 6. As shown in FIG. 7, a word is, for example, information regarding an abnormal shadow in a medical image. As a method for extracting words from a comment on findings, a known named entity extraction method using a natural language processing model such as, for example, bidirectional encoder representations from transformers (BERT) can be applied as appropriate.

Further, it is preferable that the extraction unit 32 specifies a factuality of the extracted words. The factuality means the presence or absence and the accuracy of a lesion, a property, a disease name, and the like. This is because, for example, the comments on findings include descriptions that are not certain, such as “lung adenocarcinoma is suspected”, and intentional descriptions of the lesion and the property that do not exist, such as “no spicula is found”.

The classification unit 34 classifies the words extracted by the extraction unit 32 into predetermined items (so-called “structuring”) to generate structured data of the comment on findings. Specifically, it is preferable that the classification unit 34 classifies words in the same manner as the items of the structured data (see FIG. 2) of the case recorded in the report DB 8. As a method for classifying words, for example, a dictionary in which the words that may be included in the comments on findings are classified for each item may be stored in the storage unit 22 or the like in advance, and the classification unit 34 may classify the words by collating the words extracted by the extraction unit 32 with the dictionary. Further, it is preferable that the classification unit 34 normalizes the words extracted by the extraction unit 32 to generate structured data.

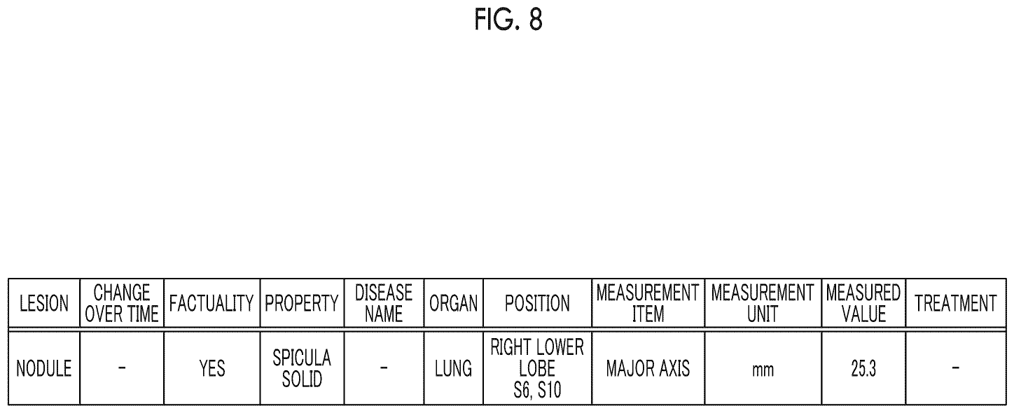

FIG. 8 shows structured data obtained by classifying words shown in FIG. 7 for each item. As shown in FIGS. 2 and 8, the predetermined item indicates, for example, at least one of a lesion (that is, the name of the abnormal shadow), a property, a disease name, a position, or a measured value included in the medical image.

The lesion is, for example, a name (type) of an abnormal shadow included in a medical image, such as “nodule”, “ground glass opacity”, and “cyst”. For example, in the case of a lung nodule, the properties are findings indicating opacity such as “solid” and “ground-glass”, margin shapes such as “well-defined/ill-defined”, “smooth/irregular”, “spicula”, “lobulated”, and “lagged”, and an overall shape such as “round” and “irregular form”. Also, for example, the properties are the relationship with the peripheral tissue, such as “pleural contact” and “pleural invagination”, and findings regarding the presence or absence of contrast, washout, and the like. The disease name is, for example, a disease name such as “cancer” and “inflammation”, and an evaluation result of “negative/positive”, “benign/malignant”, “mild/severe”, and the like regarding the disease name and the property.

The position is, for example, an anatomical position, a position in a medical image, and a relative positional relationship with other regions of interest such as “inside”, “margin”, and “periphery”. The anatomical position may be expressed by the name of an organ or tissue such as “lung” and “liver”, and may be expressed in terms of subdivided lungs such as “right lung”, “upper lobe”, and apical segment (“S1”). The measured value is a value that can be quantitatively measured from a medical image, and examples thereof include a major axis, a minor axis, a volume, a CT value whose unit is HU, the number of regions of interest in a case in which there are a plurality of regions of interest, and a distance between regions of interest. Further, the measured value may be expressed in qualitative expressions such as “larger/smaller” and “more/less”.

Further, in a case in which the factuality is specified by the extraction unit 32, the classification unit 34 may provide an item of the factuality to generate structured data. Further, the item to be included in the structured data is not limited to the above item, and other items may be added as appropriate. For example, as shown in FIGS. 2 and 8, the classification unit 34 may provide an item indicating treatment content for lesions included in the structured data to generate structured data.

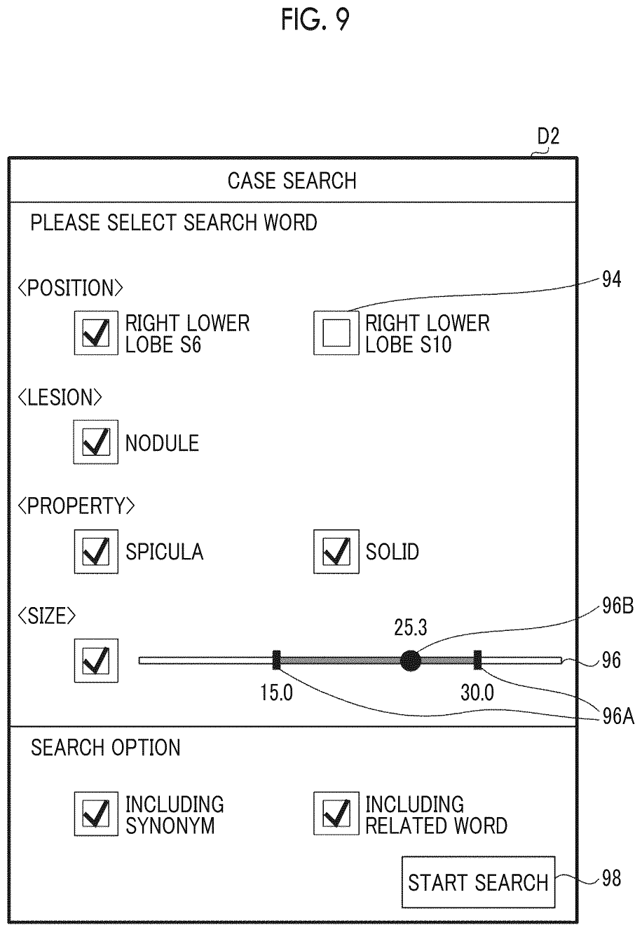

The control unit 36 performs control to display words classified by the classification unit 34 on the display 24 for each item. FIG. 9 shows an example of a screen D2 displayed on the display 24 by the control unit 36. On the screen D2, the words are classified into each item of the position, the lesion, the property, and the size (measured value) based on the structured data of FIG. 8, and are displayed.

In addition, in a case in which the plurality of words are extracted from the comments on findings by the extraction unit 32, the control unit 36 may receive selection of at least one word (hereinafter referred to as a “search word”) used for searching for a case by the search unit 38. Specifically, the control unit 36 may perform control to display an operation unit that receives selection of at least one search word used for searching for a case on the display 24 in association with the word extracted from the comment on findings. The operation unit is a part that can be optionally operated by the user on the screen displayed on the display 24, and is a graphical user interface (GUI) component, for example. On the screen D2, a check box 94 as an example of an operation unit is disposed for each word, and the user can select which word to use for the search.

Further, in a case in which the search word indicates a numerical value, the control unit 36 may receive designation of a numerical value range that is considered to be similar to the comments on findings acquired by the acquisition unit 30 (that is, included in the search result). A slider bar 96 for designating a range of nodule sizes to be included in the search result of the case is displayed on the screen D2. On the slider bar 96, two sliders 96A indicating an upper limit and a lower limit of the numerical value range and an icon 96B indicating the position of the search word “25.3 (mm)” extracted from the comment on findings are displayed. The user operates the slider 96A to designate the range of nodule sizes to be included in the search result of the case. In the example of FIG. 9, the range is designated such that the lower limit is “15.0 (mm)” and the upper limit is “30.0 (mm)”.

Further, the screen D2 includes a search start button 98. In a case in which the search start button 98 is selected by the operation of the input unit 25 by the user, the search unit 38 searches for a case similar to the content of the comments on findings acquired by the acquisition unit 30 from among the plurality of cases recorded in the report DB 8 based on the search word. For example, the search unit 38 searches for cases including the search word with reference to the structured data recorded in the report DB 8.

In addition, a check box 94 for selecting whether or not to perform the search including synonyms and related words is also displayed on the screen D2 as a search option. In response to the input of the check box 94, the search unit 38 may search for a case based on at least one of synonyms or related words predetermined for each search word. That is, the search unit 38 may search for cases based on synonyms and/or related words of the search word in addition to or instead of the search word.

The synonyms are words that have different word forms but the same or similar meanings, and in a case of “spicula”, examples thereof include “spicule”, “spinous process”, and “fluff-like”. The related words are other words that relatively often appear together in comments on findings including a certain word, and in a case of “spicula”, examples thereof includes “nodule” and “adenocarcinoma”. Synonyms and related words for each word may be stored in the storage unit 22 or the like in advance, for example.

Specifically, in a case in which a case is not sufficiently found even after searching using a search word, it is preferable to perform a search again using synonyms and/or related words. In particular, cases that include synonyms are considered to have substantially the same content as cases that include search words. Therefore, first, in a case in which the number of cases found based on the search word itself does not satisfy a predetermined threshold value, the search unit 38 may search for cases based on synonyms. Furthermore, in a case in which the number of cases found based on synonyms does not satisfy a predetermined threshold value, the search unit 38 may search for cases based on related words. Each threshold value may be stored in the storage unit 22 in advance, for example, or may be set optionally by the user.

Regarding related words, the search unit 38 may specify, as related words, a plurality of words whose degree of co-occurrence is equal to or higher than a predetermined threshold value based on a plurality of cases registered in the report DB 8. For example, in a case in which the number and/or percentage of comments on findings including “adenocarcinoma” among the plurality of comments on findings including “spicula” registered in the report DB 8 is equal to or greater than a threshold value, the search unit 38 may specify “spicula” and “adenocarcinoma” as related words.

Moreover, it is preferable that the search unit 38 searches for cases based on the factuality regardless of whether the search is performed using any of a search word, a synonym, or a related word. This is because cases with different factualities may not have similar content even in a case in which the search word, synonyms, and related words match in word form.

Furthermore, it is preferable that the search unit 38 derives a degree of similarity between the content of the comments on findings (that is, the search word) and the case. For example, the search unit 38 may derive a degree of similarity corresponding to how much the search word is included for each piece of structured data recorded in the report DB 8. Further, for example, the search unit 38 may derive a degree of similarity corresponding to how much the search word is included for each item of the structured data and derive an average value of degrees of similarity of all the items as an overall degree of similarity.

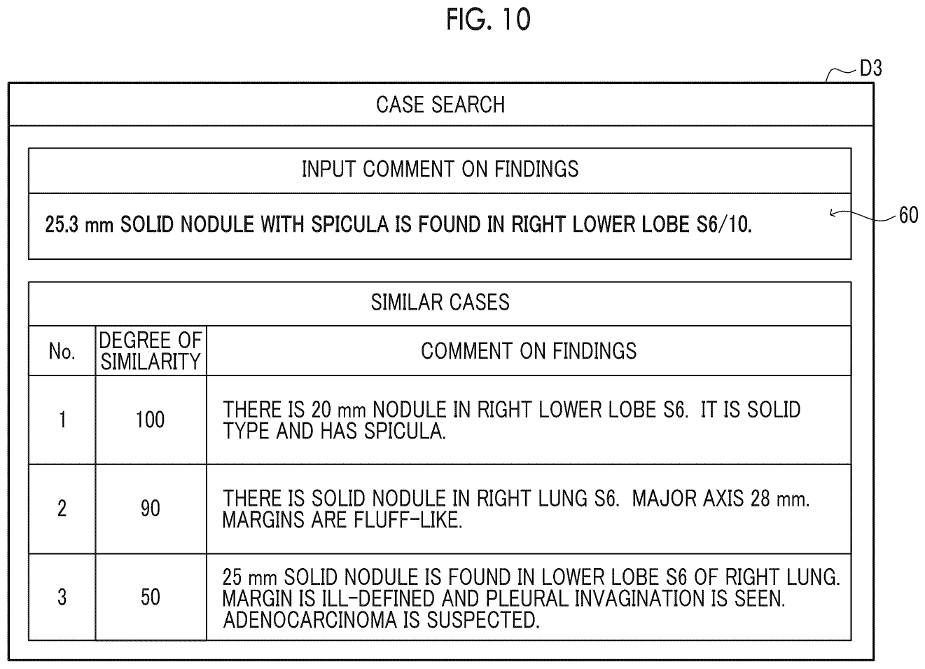

The control unit 36 performs control to display the case found by the search unit 38 and the degree of similarity derived by the search unit 38 on the display 24. FIG. 10 shows an example of a screen D3 displayed on the display 24 by the control unit 36. The screen D3 includes the comments on findings 60 used for the search and the cases (comments on findings) found by the search unit 38, which is similar to the comments on findings 60. Further, the degree of similarity with the comments on findings 60 is displayed for each case (comment on findings).

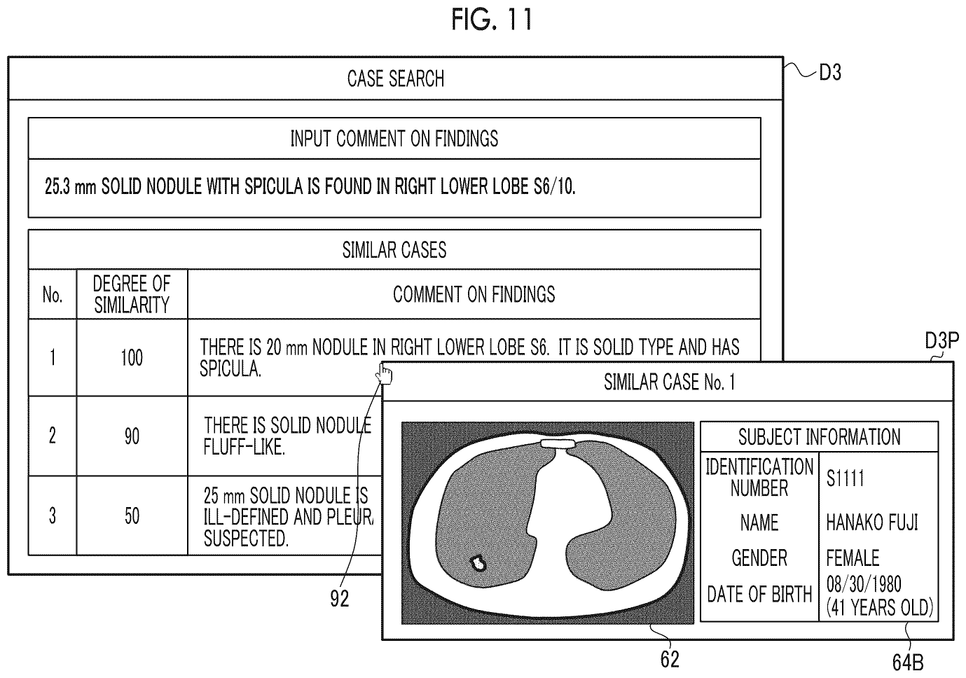

Further, the control unit 36 may perform control to display at least one of a medical image, accessory information, or biological information related to the comments on findings found by the search unit 38 on the display 24. FIG. 11 shows an example of screens D3 and D3P displayed on the display 24 by the control unit 36. The screen D3P is a pop-up screen that is displayed in a case in which the case (comment on findings) of No. 1 is selected on the screen D3. On the screen D3P, a medical image 62 related to a case (comment on findings) of No. 1 and subject information 64B as an example of accessory information attached to the medical image are displayed.

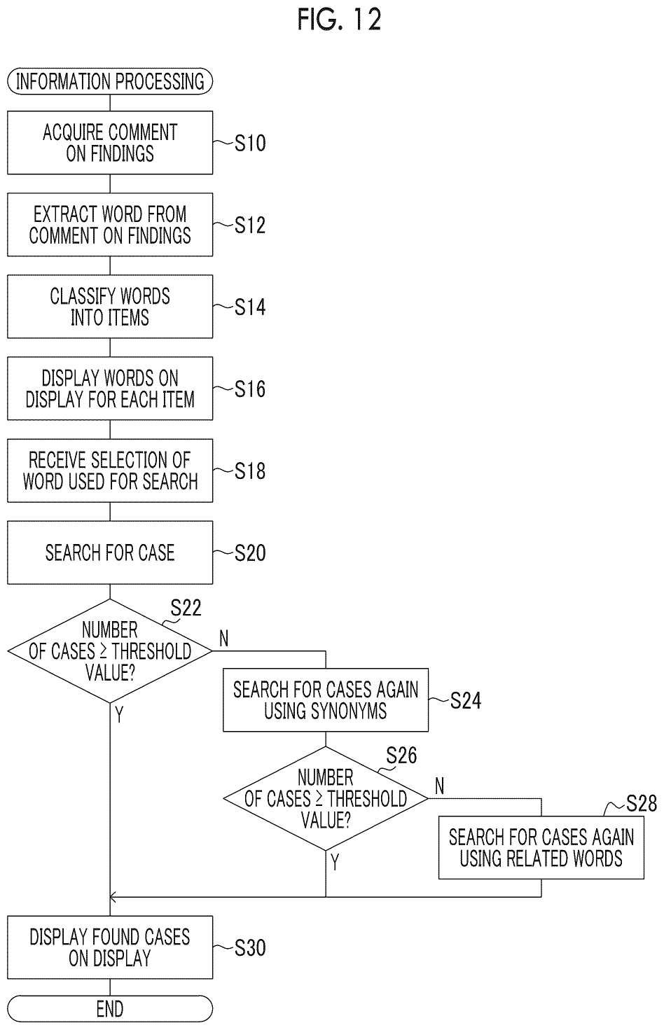

Next, with reference to FIG. 12, operations of the information processing apparatus 10 according to the present exemplary embodiment will be described. In the information processing apparatus 10, as the CPU 21 executes the information processing program 27, information processing shown in FIG. 12 is executed. The information processing is executed, for example, in a case in which the user gives an instruction to start execution via the input unit 25.

In Step S10, the acquisition unit 30 acquires at least one comment on findings. Here, the comment on findings may be input by the user via the input unit 25, or a part of the comment on findings may be selected. In Step S12, the extraction unit 32 extracts at least one word included in the comment on findings acquired in Step S10. In Step S14, the classification unit 34 classifies words extracted in Step S12 into predetermined items.

In Step S16, the control unit 36 performs control to display the words classified in Step S14 on the display 24 for each item. In Step S18, the control unit 36 receives selection of at least one word used for the search of the case from among the words displayed on the display 24 in Step S16. In Step S20, the search unit 38 searches for a case similar to the content of the comment on findings acquired in Step S10 from among the plurality of cases recorded in the report DB 8 based on the word selected in Step S18.

In Step S22, the search unit 38 determines whether or not the number of cases found in Step S20 is equal to or greater than a predetermined threshold value. In a case in which the number of cases is less than the threshold value (that is, in a case in which a negative determination is made in Step S22), the process proceeds to Step S24, and the search unit 38 searches for cases again based on the synonyms of the word selected in Step S18. In Step S26, the search unit 38 determines whether or not the number of cases found in Step S24 is equal to or greater than a predetermined threshold value. In a case in which the number of cases is less than the threshold value (that is, in a case in which a negative determination is made in Step S26), the process proceeds to Step S28, and the search unit 38 searches for cases again based on the related words of the word selected in Step S18.

On the other hand, in a case in which the number of cases is equal to or greater than the threshold value (that is, in a case in which an affirmative determination is made in Steps S22 and S26), and after Step S28, the process proceeds to Step S30. In Step S30, the control unit 36 performs control to display the cases found in Steps S20, S24, and/or S28 on the display 24 and ends the present information processing.

As described above, the information processing apparatus 10 according to one aspect of the present disclosure comprises at least one processor. The processor extracts at least one word included in a comment on findings, classifies the word into a predetermined item, displays the word for each item on a display, and searches for medical information similar to content of the comment on findings from among a plurality of pieces of recorded medical information based on the word.

In other words, with the information processing apparatus 10 according to the present exemplary embodiment, it is possible to search for a case based on the comments on findings. Further, by displaying the words extracted from the comments on findings on the display for each item, the content of the comments on findings and the search words can be easily confirmed. Therefore, for example, the time and effort such as the input of the search word and the setting of the search condition can be omitted, and the cases can be easily found.

Note that, in the above exemplary embodiment, the form has been described in which the comments on findings used for the search are manually input by the user such as the radiologist, but the present disclosure is not limited thereto. The comments on findings used for the search may be, for example, comments on findings included in the interpretation report recorded in the report DB 8, the storage unit 22, or the like. Further, for example, the comment on findings may be generated using machine learning based on a medical image acquired by the acquisition unit 30. As a method for generating comments on findings using machine learning, for example, a method using a recurrent neural network described in JP2019-153250A can be applied as appropriate.

Further, in the exemplary embodiment, the form has been described in which the medical image, the imaging information, the subject information, the biological information, and the like are displayed on the display 24 as the medical information for creating the interpretation report (see FIG. 5), but the present disclosure is not limited thereto. For example, by displaying a detection result and/or a diagnosis result of a lesion obtained by analyzing a medical image using a known CAD technology on the display 24, the control unit 36 may support the user to input the comments on findings with reference to the detection result and/or the diagnosis result.

Further, in the above exemplary embodiment, as an example of a case (medical information), the description has been given using the interpretation report, the comments on findings, and the structured data recorded in the report DB 8, but the present disclosure is not limited thereto. A case may be, for example, medical information indicating at least one of a medical image, a comment on findings regarding the medical image, subject information regarding a subject of the medical image, or biological information acquired from the subject. Specifically, the case may be a medical image recorded in the image DB 6, accessory information of the medical image (subject information and imaging information), or the like. Furthermore, for example, the biological information may be biological information recorded in the storage unit 22, the image DB 6, the report DB 8, an external device (not shown), or the like.

Further, in the above exemplary embodiment, the form has been described in which the comments on findings are displayed on the display 24 as the search result of the case (see FIG. 10), but the present disclosure is not limited thereto. For example, the control unit 36 may perform control to display various types of medical information such as structured data, a medical image, subject information, and biological information on the display 24 as a search result of the case.

Further, in the above exemplary embodiment, the form has been described in which the case is found based on the search word and the structured data, but the present disclosure is not limited thereto. For example, the search unit 38 may perform the search of the case, the derivation of the degree of similarity, or the like based on an image feature amount of the medical image. Specifically, the search unit 38 may search for the medical image including the image feature amount meant by the search word from among the medical images recorded in the image DB 6. The image feature amount may be derived using, for example, a learning model such as a convolutional neural network (CNN) that has been trained in advance such that an input is a medical image and an output is an image feature amount of the medical image.

In addition, in the above exemplary embodiment, the form has been described in which the search is performed using the search words described in the comments on findings first and then the search is performed using the synonyms and then the related words, but the present disclosure is not limited thereto. For example, in a case in which “including synonym” and/or “including related word” is selected on the screen D2 of FIG. 9, the synonym and/or the related word may be unconditionally used for the search.

Further, in the above exemplary embodiment, the form has been described in which the search is performed based on the comments on findings selected from among the plurality of comments on findings, but the present disclosure is not limited thereto. For example, in a case in which a sufficient number of cases cannot be found by performing the search based on the selected comments on findings, the search may be performed using words included in other comments on findings that are not selected, and synonyms and related words thereof. For example, in the case of FIG. 6, the case may be found based on the unselected comment on findings “A tumor with a diameter of 4 cm is found in the right internal deep neck region”.

Specifically, in a case in which the number of cases found by the search unit 38 based on at least one of the search word, the synonym, or the related word does not satisfy a predetermined threshold value, the extraction unit 32 may extract at least one word included in another comment on findings related to the comment on findings selected from among the plurality of comments on findings. The classification unit 34 may classify words extracted by the extraction unit 32 into predetermined items. The search unit 38 may search for medical information based on words extracted from another comment on findings.

In addition, in the above exemplary embodiment, in a case in which a plurality of search words are selected, the search unit 38 may search for cases based on the weight predetermined for each item, such as the lesion, the property, the position, and the measured value. Specifically, in a case in which the weight of an n-th item is denoted by wn and the degree of similarity between the search word and the case in the n-th item is denoted by sn, the search unit 38 may search for cases using an overall degree of similarity x expressed by the following formula. The formula below is a weighted arithmetic average formula.

$$x = ( w 1 s 1 + w 2 s 2 + … + wnsn ) / n$$

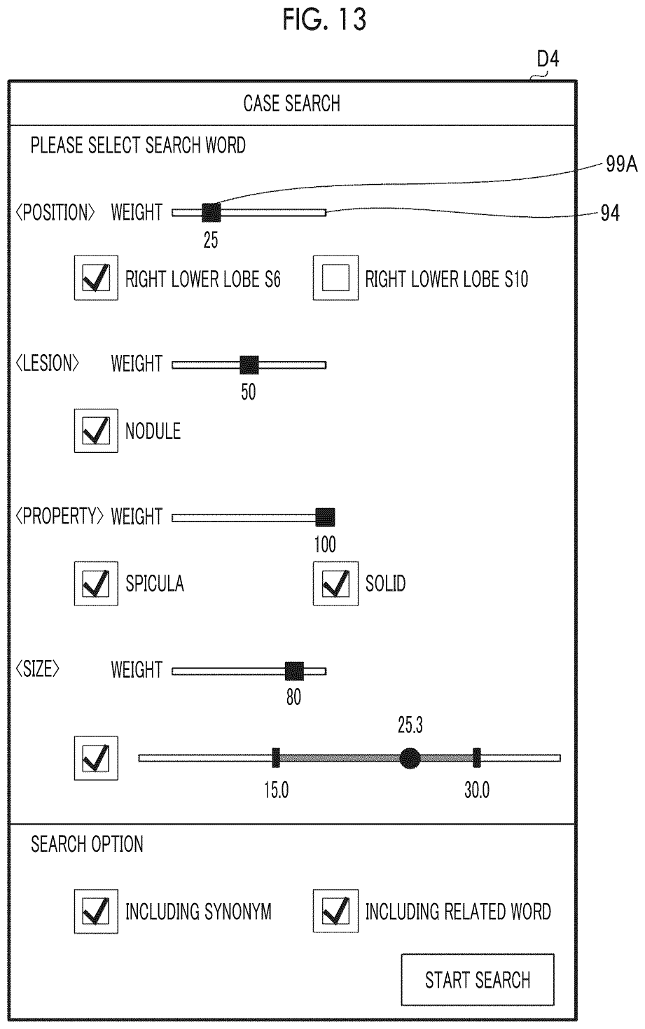

In this way, by searching for cases while taking into consideration weights, it is possible to search for cases in which the items particularly emphasized by the user are similar. Note that the weight for each item may be set in advance and stored in the storage unit 22, for example, or may receive a setting by the user. FIG. 13 shows an example of a screen D4 for receiving a setting of the weight displayed on the display 24 by the control unit 36. On the screen D4, in addition to the screen D2 of FIG. 9, a slider bar 99 for setting the weight is displayed for each item. The user sets the weight of each item by operating the slider 99A on the slider bar 99.

In the above exemplary embodiment, for example, as hardware structures of processing units that execute various kinds of processing, such as the acquisition unit 30, the extraction unit 32, the classification unit 34, the control unit 36, and the search unit 38, various processors shown below can be used. As described above, the various processors include a programmable logic device (PLD) as a processor of which the circuit configuration can be changed after manufacture, such as a field-programmable gate array (FPGA), a dedicated electrical circuit as a processor having a dedicated circuit configuration for executing specific processing such as an application-specific integrated circuit (ASIC), and the like, in addition to the CPU as a general-purpose processor that functions as various processing units by executing software (program).

One processing unit may be configured by one of the various processors, or may be configured by a combination of the same or different kinds of two or more processors (for example, a combination of a plurality of FPGAs or a combination of the CPU and the FPGA). In addition, a plurality of processing units may be configured by one processor.

As an example in which a plurality of processing units are configured by one processor, first, there is a form in which one processor is configured by a combination of one or more CPUs and software as typified by a computer, such as a client or a server, and this processor functions as a plurality of processing units. Second, as represented by a system-on-chip (SoC) or the like, there is a form of using a processor for realizing the function of the entire system including a plurality of processing units with one integrated circuit (IC) chip. In this way, various processing units are configured by one or more of the above-described various processors as hardware structures.

Furthermore, as the hardware structure of the various processors, more specifically, an electrical circuit (circuitry) in which circuit elements such as semiconductor elements are combined can be used.

In the above exemplary embodiment, the information processing program 27 is described as being stored (installed) in the storage unit 22 in advance; however, the present disclosure is not limited thereto. The information processing program 27 may be provided in a form recorded in a recording medium such as a compact disc read-only memory (CD-ROM), a digital versatile disc read-only memory (DVD-ROM), and a universal serial bus (USB) memory. In addition, the information processing program 27 may be configured to be downloaded from an external device via a network. Further, the technology of the present disclosure extends to a storage medium for storing the information processing program non-transitorily in addition to the information processing program.

The technology of the present disclosure can be combined as appropriate with the above exemplary embodiment. The described contents and illustrated contents shown above are detailed descriptions of the parts related to the technology of the present disclosure, and are merely an example of the technology of the present disclosure. For example, the above description of the configuration, function, operation, and effect is an example of the configuration, function, operation, and effect of the parts related to the technology of the present disclosure. Therefore, needless to say, unnecessary parts may be deleted, new elements may be added, or replacements may be made to the described contents and illustrated contents shown above within a range that does not deviate from the gist of the technology of the present disclosure.

The disclosure of JP2022-024250 filed on Feb. 18, 2022 is incorporated herein by reference in its entirety. All documents, patent applications, and technical standards described in the present specification are incorporated in the present specification by reference to the same extent as in a case in which each of the documents, patent applications, technical standards are specifically and individually indicated to be incorporated by reference.

What is claimed is:

1. An information processing apparatus comprising at least one processor, wherein the processor is configured to:
extract at least one word included in a comment on findings;
classify the word into a predetermined item;
display the word for each item on a display; and
search for medical information similar to content of the comment on findings from among a plurality of pieces of recorded medical information based on the word.

2. The information processing apparatus according to claim 1, wherein the processor is configured to, in a case in which a plurality of the words are extracted from the comment on findings, receive selection of at least one of the words used for searching for the medical information.

3. The information processing apparatus according to claim 2, wherein the processor is configured to display an operation unit that receives the selection of at least one of the words used for searching for the medical information on the display in association with the word.

4. The information processing apparatus according to claim 1, wherein the processor is configured to search for the medical information based on at least one of a synonym or a related word predetermined for each word.

5. The information processing apparatus according to claim 4, wherein the processor is configured to, in a case in which the number of pieces of the medical information found based on the word does not satisfy a predetermined threshold value, search for the medical information based on the synonym.

6. The information processing apparatus according to claim 5, wherein the processor is configured to, in a case in which the number of pieces of the medical information found based on the synonym does not satisfy a predetermined threshold value, search for the medical information based on the related word.

7. The information processing apparatus according to claim 4, wherein the processor is configured to specify a plurality of words whose degree of co-occurrence is equal to or higher than a predetermined threshold value, based on the plurality of pieces of medical information, as related words.

8. The information processing apparatus according to claim 1, wherein the processor is configured to:
acquire a plurality of the comments on findings; and
receive selection of at least one of the plurality of comments on findings used for searching for the medical information.

9. The information processing apparatus according to claim 8, wherein the processor is configured to, in a case in which the number of pieces of the medical information found based on the word does not satisfy a predetermined threshold value:
extract at least one word included in another comment on findings related to the comment on findings selected from among the plurality of comments on findings; and
search for the medical information based on the word extracted from the other comment on findings.

10. The information processing apparatus according to claim 1, wherein the processor is configured to search for the medical information based on a weight predetermined for each item.

11. The information processing apparatus according to claim 10, wherein the processor is configured to receive a setting of the weight for each item.

12. The information processing apparatus according to claim 1, wherein the processor is configured to:
specify factuality of the extracted word; and
search for the medical information based on the factuality.

13. The information processing apparatus according to claim 1, wherein the processor is configured to, in a case in which the word indicates a numerical value, receive designation of a numerical value range that is considered to be similar to the comment on findings.

14. The information processing apparatus according to claim 1, wherein:
the word indicates information regarding an abnormal shadow in a medical image, and
the item indicates at least one of a name, a property, a disease name, a position, or a measured value of the abnormal shadow.

15. The information processing apparatus according to claim 1, wherein the processor is configured to display the found medical information on the display.

16. The information processing apparatus according to claim 1, wherein the processor is configured to:
derive a degree of similarity between the content of the comment on findings and the medical information; and
display the derived degree of similarity on the display.

17. The information processing apparatus according to claim 1, wherein the medical information indicates at least one of a medical image, a comment on findings regarding the medical image, subject information regarding a subject of the medical image, or biological information acquired from the subject.

18. The information processing apparatus according to claim 17, wherein the medical information indicates the comment on findings.

19. An information processing method comprising:
extracting at least one word included in a comment on findings;
classifying the word into a predetermined item;
displaying the word for each item on a display; and
searching for medical information similar to content of the comment on findings from among a plurality of pieces of recorded medical information based on the word.

20. A non-transitory computer-readable storage medium storing an information processing program for causing a computer to execute a process comprising:
extracting at least one word included in a comment on findings;
classifying the word into a predetermined item;
displaying the word for each item on a display; and
searching for medical information similar to content of the comment on findings from among a plurality of pieces of recorded medical information based on the word.
